(19) United States
(12) Patent Application Publication
(10) Pub. No.: US 2018/0228194 A1
(43) Pub. Date: Aug. 16, 2018

(54) FUNCTIONAL WINE

(71) Applicants: RAFAEL FRANCISCO IÑIGO PAVLOVICH, HERMOSILLO (MX); ALAN GUEVARA MORALES, HERMOSILLO (MX)

(72) Inventors: RAFAEL FRANCISCO IÑIGO PAVLOVICH, HERMOSILLO (MX); ALAN GUEVARA MORALES, HERMOSILLO (MX)

(21) Appl. No.: 15/430,415

(22) Filed: Feb. 10, 2017

(51) Int. Cl. A23L 33/105; A61K 36/67; C12G 3/02; C07C 39/21

(52) U.S. Cl. CPC A23L 33/105; A61K 36/67; C12G 3/02; A23V 2250/21; C12G 2200/21; A23V 2002/00; C07C 39/21

(57) ABSTRACT

This invention comprises a new mixture aimed to maximize in the health of the human body the positive effects of polyphenols contained in fruits such as grapes, blueberries, and blackberries, and also in products derived from them such as jams and red wines. The mixture includes, specifically, the use of a standardized extract of black pepper, of the piper nigrum species. This extract is diluted in a drink, generally wine, whose content includes polyphenols. The addition of the extract to the wine in set proportions and its subsequent consumption entail an increase in the bioavailability of the main polyphenol included in wine, resveratrol. The increase of the bioavailability generates a series of positive effects in the body such as prevention of cardiovascular and neurodegenerative disorders, anti-cancer properties, anti-diabetic effects, and against osteoporosis.

### CROSS-REFERENCE TO RELATED APPLICATIONS

This application claims the benefit of PPA Ser. No. 62/294,098, filed Feb. 11, 2016 by the present inventors, which is incorporated by reference.

### FIELD OF THE INVENTION

This invention, as it is mentioned in the title of this descriptive report, falls within biochemistry field. Particularly to a wine composition and to a substance that allows the absorption on the human body of the polyphenols contained in wine. Specifically, to a wine mixed with a black pepper (piper nigrum) extract, which facilitates the absorption of resveratrol, slowing down its metabolism time to increase its absorption in the bloodstream after its oral ingestion.

### BACKGROUND

Polyphenols are the most abundant antioxidant substances in the human diet. Resveratrol is among the polyphenols that shows beneficial effects for the heart, and has other qualities such as anti-cancer activity, anti-diabetic effects, and against osteoporosis, also helping in preventive treatment of neurodegenerative disorders.

Resveratrol is included in fruits such as grapes, blueberries, and blackberries, as well as in products derived from them such as jams and red wines.

Despite the positive characteristics of resveratrol, its assimilation within the human body is reduced because of its fast metabolism that takes place during the first hour after its oral administration. The foregoing because of a glucuronidation process in the trans-resveratrol metabolites, being this the primary configuration and the most studied, where metabolites are quickly eliminated through the urinary tract.

Document U.S. Pat. No. 6,054,585 describes a process to prepare high purity piperine through the removal of the impurities present in the oleoresin of the fruit or plant of the Piperaceae family or any other natural source of the alkaloid piperine. The product obtained is 98% pure, and the recommended dose ranges between 0.0004 to 0.15 milligrams per kilogram per day. Specifically, the dose recommended for humans is 5 milligrams per day per person, which can increase up to 15 milligrams per day per person for treatments due to nutritional shortfalls.

Document U.S. Pat. No. 5,536,506 refers to a new composition and method to improve gastrointestinal absorption and systematic use of nutrients and nutritional supplements, where the composition includes an extract of the pepper fruit which consists of a minimum of 98% of pure piperine alkaloid. The method involves the oral, topical, or parenteral administration of the invention compositions. Also, a new procedure for piperine extraction and purification is described.

Document ES2161914 describes the use of piperine, or of an extract that contains piperine, for the fabrication of a composition aimed at increasing the gastrointestinal absorption of nutrients in a person at a dose of 0.15 milligrams, or less, per kilogram of body weight of the person.

However, in the market, the administration of high purity piperine involves the use of food supplements, generally in capsules. The solution claimed in this descriptive report differs of what is currently available, because it proposes the use of the same nutrient vehicle that shows deficiencies in nutrient absorption to transport the high purity piperine into the body, where it will facilitate the absorption of said nutrients.

### DETAILED DESCRIPTION OF THE INVENTION

The invention described in this descriptive report proposes the use of the substance that carries the nutrients as transport for the administration of the component, which will facilitate the absorption of said nutrients, by making it a unique product in its category.

The invention includes a new wine composition to make that the polyphenols contained in said liquid are more easily absorbed when crossing the digestive tract, especially in the small intestine.

Since wine contains many polyphenols, especially resveratrol, whose properties are appreciated in the preventive medicine sector, resveratrol can be used to transport substances that allow improving the absorption of said resveratrol. This polyphenol presents certain difficulty to be absorbed by the intestine and to be transferred to the bloodstream for the utilization of its properties. Diverse studies show that piperine increases the permeability of the intestinal wall, allowing the pass of resveratrol and the increment of its bioavailability in the human body.

The objective reached by this invention, that is to say, the increase of polyphenols bioavailability contained in the wine in the bloodstream, specifically, resveratrol, arises from the use of piperine contained in a standardized extract to 98% of piper nigrum along with a common wine. The extract is odorless, tasteless, and does not affect the wine organoleptic test once they are mixed, that is, it does not change the wine sensory perception, and this can be consumed appreciating its original body, flavor, and odor.

The addition of said extract to the wine allows that the intestine is permeable to the pass of the polyphenols naturally contained in the wine, specifically resveratrol, thus allowing its assimilation and utilization of its preventive qualities, by way of example and without limitation, cardiovascular and neurodegenerative disorders.

### PREFERRED EMBODIMENT OF THE INVENTION

The mixture of the piper nigrum extract with the wine must be done in set proportions in order to prevent an extract overdose. Specifically, the extract is mixed in the range between 15 and 30 milligrams per 750 milliliters of a wine with polyphenols content; this is because the recommended administration is of 5 milligrams per dose, where each dose includes approximately a glass of wine.

Assuming a moderate consumption, each bottle of wine, containing 750 milliliters of drink mixed with 25 milligrams of piper nigrum extract, will contain in turn 5 doses of functional wine.

Even though in this descriptive report the invention is dissolved in wine, it is important to clarify that any other drink containing polyphenols, especially resveratrol, will be susceptible to be mixed as indicated in order to increase the bioavailability of said polyphenols in the bloodstream, enhancing positive health benefits.

Having described my invention in a sufficient and clear manner, I consider it as a novelty; therefore, I claim as my exclusive property what is contained in the following clauses:

1. A functional wine that comprises the mixture of:
a wine that includes at least one polyphenol; and
an extract of black pepper of the piper nigrum species;
wherein the pepper extract is a standardized extract to 98% purity, odorless, tasteless, and does not affect the wine organoleptic test.

2. The functional wine of claim 1, wherein the polyphenol contained in the wine is resveratrol (3,5,4-trihidroxistilbene).

3. The functional wine of claim 1, wherein the mixture contains a concentration equal to, or lower than, 0.04 grams per liter.

4. The functional wine of claim 1, wherein the mixture is made adding between 15 and 30 milligrams of an extract of black pepper, of the piper nigrum species, per each 750 milliliters of wine.
